(19) United States
(12) Patent Application Publication
(10) Pub. No.: US 2004/0241693 A1
(43) Pub. Date: Dec. 2, 2004

(54) Method for moving a fluid of interest in a capillary tube and fluidic microsystem

(76) Inventors: Florence Ricoul, Quaix-en-Chauteuse (FR); Jean Berthier, Meylan (FR); Jerome Boutet, Fontaine (FR)

Correspondence Address: PEARNE & GORDON LLP, 1801 EAST 9TH STREET, SUITE 1200, CLEVELAND, OH, 44114-3108, US

(21) Appl. No.: 10/488,435

(22) PCT Filed: Sep. 19, 2002
(86) PCT No.: PCT/FR02/03207

(30) Foreign Application Priority Data
Sep. 21, 2001 (FR) 01/12192

(51) Int. Cl. C12Q 1/68; C12M 1/34

(52) U.S. Cl. 435/6; 435/287.1

(57) ABSTRACT

The present invention relates to a method for the displacement of an analyte fluid within a capillary microchannel and to a microfluidic system. In particular, it relates to the field of microfluidics, and especially to microfluidic systems. The method comprises steps which consist in introducing at least one ferrofluid train (3) into the said capillary channel (1), the said ferrofluid train (3) comprising a slug of ferrofluid (5) and, placed against at least one of the two ends of the slug of ferrofluid and in contact with it, a slug of liquid (7) immiscible with both the ferrofluid and the analyte fluid; in introducing the said analyte fluid (9) into the said capillary channel, in proximity to the ferrofluid train and on the side having the slug of liquid (7) immiscible with both the ferrofluid and the analyte fluid; and in controlling the analyte fluid displacement within the said capillary channel by the action of a magnetic field on the ferrofluid train, which field is generated by a magnet system placed on the outside of the said capillary channel.

### TECHNICAL FIELD OF THE INVENTION

The present invention relates to a method for the displacement of an analyte fluid within a capillary channel and to a microfluidic system.

It relates in particular to the field of microfluidics, and especially to microfluidic systems. It makes it possible to perform chemical or biological processes with high throughputs.

By using microtechnology process techniques it also allows for integration into devices currently referred to as “lab-on-a-chip” and also “micro-Total-Analysis-Systems” or MicroTAS.

In the lab-on-a-chip example, the present invention may be combined with other functions in order to form a more complete and more accurate biological analysis system.

### PRIOR ART

The development and utilisation of microfluidic systems for obtaining chemical or biological analytical information has shown continuous growth in recent years.

One of the most serious problems to be solved for the implementation of this novel microchannel technology is the issue of how to control fluid flow or transport within the microchannels.

In addition, the increase in analyser flow rates could require the stacking in series along the microchannels of several different reactive liquids in the form of slugs, which then adds the problem of biological contamination of one slug by another.

Certain techniques in the prior art propose the use of variable surface states as a means of flow regulation whilst, however, imposing certain constraints on the physicochemical properties of the fluids to be transported and a precisely defined treatment of the surfaces. It is also possible to use the generation of bubbles for flow regulation within capillary channels. Finally, mechanical systems for regulating the hydrostatic pressure also exist; these can be fitted upstream of the microcircuits or downstream, for example by the installation of a wick made of absorbent material.

Unfortunately, apart from the lack of precision of these systems, and the difficulty of implementing them, none of them solves the aforementioned problems of the prior art.

### DESCRIPTION OF THE INVENTION

One object of the present invention is precisely to provide a solution to the aforementioned problems of the prior art by providing a method for the displacement of an analyte fluid within a capillary channel comprising the following steps:

at least one ferrofluid train is introduced into the said capillary channel, the said ferrofluid train comprising one slug of ferrofluid and, placed at least at one of the two ends of the slug of ferrofluid and in contact with it, a slug of liquid immiscible with both the ferrofluid and the analyte fluid,

the said analyte fluid is introduced into the said capillary channel in proximity to the ferrofluid train, and

the displacement of the analyte fluid within the said capillary channel is controlled by the action of a magnetic field on the said slug of ferrofluid, which field is generated by a magnet system placed on the outside of the said capillary channel.

A further object of the present invention is to provide a microfluidic system for the displacement of an analyte fluid comprising, on the one hand, a capillary channel into which at least one ferrofluid train is introduced and, on the other hand, external to the said capillary channel, a magnet system capable of producing a magnetic field for controlling the displacement of the ferrofluid train within the capillary channel, the said train comprising a slug of ferrofluid and, placed against at least one of the two ends of the slug of ferrofluid and in contact with it, a slug of liquid immiscible with both the ferrofluid and the analyte fluid.

By analyte fluid we mean any liquid or gaseous fluid which it is necessary to displace within a capillary channel, for example in microfluidic systems. The analyte fluid could be, for example, a chemical reagent, a biological liquid, an aqueous solution, etc.

By slug we mean a volume of fluid within the capillary channel forming a “cylinder” which, by a capillary effect, takes the shape of the internal capillary channel wall. In other words, the fluid introduced into the capillary channel forms a slug when it fills the total cross section of the capillary channel over a length that depends on the volume of this fluid.

A ferrofluid train, also called “train” in the present description, comprises a slug of ferrofluid and at least one slug of a liquid immiscible and in contact with both the ferrofluid and the analyte fluid. The ferrofluid train moves as a whole with the plug or plugs of liquid immiscible with the ferrofluid and the analyte fluid. Various embodiments of the present invention are described below by way of examples.

Ferrofluids or magnetic fluids were discovered in the sixties and are fluids composed of essentially two constituents:

(1) single-domain particles of a ferromagnetic substance, magnetite or maghemite, about 5 to 10 nm in size,

(2) a carrier fluid.

When the carrier fluid is an organic compound, as is the case of most commercial ferrofluids, the ferrofluid is the said to be “organic-based” and the magnetic particles are dispersed in the carrier fluid by surfactants. When the carrier fluid is water, the ferrofluid is the said to be “ionic-based” and the particles are dispersed either by electrostatic forces or by surfactant bilayers.

The choice of ferrofluid corresponds to the inventor's choice of control, or drive, by a magnetic field for carrying out the method of the present invention.

Ferrofluids that can be employed for the purposes of the invention preferably have a low viscosity and good physical and chemical stability over time and as a function of temperature.

According to the invention, the ferrofluid is preferably an ionic ferrofluid, for instance a ferrofluid such as those described in document GB-A-2 244 987. These ferrofluids exhibit a high particle density and a high magnetic susceptibility, and are very stable over time. They are obtained by fixing, to the surface of the precursor magnetic particles, charged molecules that ensure colloidal stability without using surfactants.

In microanalysis systems, the analyte fluid usually takes the form of an aqueous solution. On the face of it, in lab-on-a-chip microchannels or microtubes the simplest solution for the implementation of ferrofluids according to the invention is to work with organic-based ferrofluids, since they are immiscible with water. But then the problem of contaminating and non-biocompatible deposits arises, for example deposits in the form of iron-oxide-based magnetic particles, which can interfere with the chemical reactions involved.

These deposits have been observed by the inventors in capillary channels made of glass, such as fused silica, which is somewhat hydrophilic, as well as in capillary channels whose internal walls are very hydrophobic such as Teflon (registered trademark) or Tefzel (registered trademark), for example for fluid displacement velocities as low as 0.1 mm/s. Moreover, the thickness of the contamination from the ferrofluid measured on the internal capillary channel wall is of the order of one micron, and therefore for fluid displacements of several centimeters the loss of material from the fluid slugs onto the walls can be significant. Either the presence of surfactants in these ferrofluids or the apolar nature of the carrier fluid could explain this phenomenon.

The inventors have demonstrated that the preferred combination of a slug of ionic ferrofluid, of a slug of liquid immiscible with both the ferrofluid and the analyte fluid, and preferably of a hydrophobic capillary channel wall, according to the present invention, unexpectedly provides a solution to the aforementioned problems. Indeed, laboratory tests have demonstrated the absence of any contaminating film on the internal capillary channel wall by implementing the present invention.

Thus, according to the invention, the capillary channel is preferably one having a hydrophobic internal wall, that is the internal wall has a contact angle greater than 90°. This can be achieved, for example, by an appropriate surface chemical treatment such as silanization, or by the use of hydrophobic materials such as the aforementioned ones. The capillary channel material may be chosen, for example, according to the nature of the analyte fluid and the physical and chemical conditions for the chemical reactions taking place in the capillary channel. According to the invention, the capillary channel, microtube, or microchannel, can for example have diameters of less than 1 mm, for example 0.5 mm or lower, which corresponds to the typical dimensions found in microfluidic systems.

The liquid immiscible with both the ferrofluid and the analyte fluid may be oil for example, especially when the ferrofluid is an ionic ferrofluid and the analyte fluid is an aqueous solution. The oil may be an organic oil, such as dodecane for example, or it may be a mineral oil, such as the oil M3516 marketed by Sigma-Aldrich for example.

It is to be expected that a thin film of oil will be deposited on the internal wall of the capillary channel during displacement of the ferrofluid train since oil wets a hydrophobic surface better than water. However, this will not be an issue if the oil is compatible with the analyte fluid. Thus, according to the invention it is advantageous to use a biocompatible oil, such as a mineral oil, when the analyte fluid is a biological liquid.

According to the invention, in order to work with oil buffer-slugs of minimal size, with no risk of loss of material to the microchannel walls, a step of prewetting the walls may be performed by firstly allowing a column of oil of sufficient volume to circulate within the system. Thus, according to the invention, a step of prewetting the internal capillary channel walls with oil can be performed prior to introducing the ferrofluid train into the said capillary channel.

According to the invention, individual oil slugs can also be introduced into the capillary channel without ferrofluid, for example in order to separate two slugs of identical or different analyte fluids situated between two ferrofluid trains, either before or after a single ferrofluid train. Thus, according to the present invention, at least one slug of liquid immiscible with both the ferrofluid and the analyte fluid can be introduced into the capillary channel in between two slugs of analyte fluids.

According to a first embodiment of the present invention, the ferrofluid train may be composed of one ferrofluid slug and one slug of liquid immiscible with both the ferrofluid and the analyte fluid. This embodiment is, for example, useful for displacing an analyte fluid placed on one side of the ferrofluid train only, that is on the side of the immiscible liquid.

According to a second embodiment of the present invention, a slug of liquid immiscible with both the ferrofluid and the analyte fluid is placed at each of the two ends of the ferrofluid slug. Thus, in this embodiment, the ferrofluid train comprises one ferrofluid slug and two slugs of liquid immiscible with both the ferrofluid and the analyte fluid. This embodiment is, for example, useful for displacing an analyte fluid placed on either side of the ferrofluid train, or for two different analyte fluids separated by the ferrofluid train.

According to a third embodiment of the present invention, a plurality of ferrofluid trains can be introduced into the capillary channel with either identical ferrofluids or ferrofluids that differ from one train to another, and with slugs of liquid immiscible with both the ferrofluid and the analyte fluid being either identical or different in a given train or from one train to another. This embodiment is, for example, useful for displacing several slugs of one or more identical or different analyte fluids, each slug of analyte fluid being separated from the next either by a ferrofluid train according to the present invention or by a single slug of liquid immiscible with both the ferrofluid and the analyte fluid.

Further embodiments of the present invention will be apparent to those skilled in the art.

According to the invention, the magnet system required for displacing the analyte fluid through the capillary channel, in other words for driving the flow of this fluid, may be formed by permanent magnets for example or by electrical circuits, that is by electromagnets placed, for example, in close proximity to the capillary channels. This magnet system can be fixed or mobile.

The magnetic field may be moved, for example, by mechanical displacement of a permanent magnet or electromagnet along the capillary channel, or by sequentially “activating” adjacent electromagnetic coils. The permanent magnet may be in the form of a bar magnet for example, and the electromagnet in the form of a coil or solenoid for example.

The sizes of the ferrofluid slugs and of the magnets are adapted to the conditions of the desired application of the method of the present invention, that is to say, for example, to the velocity of the fluid or to the radius of the capillary channel, so as to allow good coupling between magnet and ferrofluid slug and therefore good flow control. As an example according to the present invention, the magnets may be between 0.5 and 2 mm in length and the slugs of ferrofluid about twice that length.

The number of magnet systems can depend on the number of ferrofluid trains used. Accordingly, n fluid trains could require n magnet systems.

It can also depend on the type of control used according to the method of the invention to displace the analyte fluid.

A person skilled in the art could easily adapt the microfluidic system of the present invention to suit his needs.

Indeed, according to the present invention, the displacement of the analyte fluid inside the said capillary channel by the action of a magnetic field, generated by the magnet system placed on the outside of the said capillary channel, on the said ferrofluid slug may be controlled in various ways.

For example, the flow or displacement of the analyte fluid through the microchannel may be achieved by the driving force of pressure or suction applied within the capillary channel. In this case, the control of analyte fluid displacement in the present invention may consist in either blocking, or in allowing, fluid movement within the capillary channel by blocking, or alternatively allowing, the displacement of the ferrofluid train using the magnet system. This may be achieved by, for example, using a ferrofluid train consisting of one slug of ferrofluid with two buffer slugs of oil at each end and a single permanent magnet or electromagnet. Retracting the permanent magnet or switching off the power supplying the electromagnet allows the analyte fluid to flow again.

As a further example of the application of the method of the present invention using n steps, there are n ferrofluid slugs having 2×n buffer slugs of oil and m magnets or electromagnets, where m<n. Extra slugs of oil, without a slug of ferrofluid, allow slugs of biochemical reagents to be isolated from one another. In this configuration, flow is brought to a halt sequentially every time that a ferrofluid slug passes under a magnet. The number n will depend both on the application and the technology in question, for example the microchannel length, the multiplexing, lateral injection, etc. The greater the number m, the less magnetic force per magnet will be required, which can be a significant factor where miniaturization of the magnets is sought.

For example, another application of the present invention, called “continuous flow mode”, with or without pressure as external driving force, the microfluidic system may comprise one or n ferrofluid slugs having, respectively, one or 2×n buffer slugs of oil and a travelling magnetic field obtained either by mechanical displacement of a permanent magnet along the capillary channel or by sequentially “activating” adjacent electromagnet coils. In this example, the displacement of the magnetic field provides the driving force for the ferrofluid train, and therefore for the analyte fluid, within the capillary channel.

Thus, according to the present invention, various methods may be envisaged for controlling, or driving, analyte flow within the capillary channel or microchannels.

Furthermore, the present invention has the advantages of employing an analyte fluid displacement control or drive system external to the capillary channel, of reducing or eliminating ferrofluid deposits in the form of a liquid film on the internal capillary channel walls, and of avoiding the contamination problems associated with the prior art devices. Moreover, it offers an accurate and readily implemented method for the control of fluid flow within microchannels.

The present invention may be advantageously employed, for example, in an automated in vitro diagnostic system, or a biological contaminant detection system in such fields as the agrifood industry and/or industrial microbiological monitoring.

By way of an example, the device of the present invention may be the first element in a complete system comprising:

1. a device for the displacement of an analyte fluid according to the present invention;

2. optionally, an amplification module of the “Polymerase Chain Reaction” (PCR) type;

3. a separation module, for example using electrophoresis;

4. a detection module.

An example of an integrated device comprising the above elements 2 to 4 is described in the reference: Burns, M. A. et al, An Integrated Nanoliter DNA Analysis Device, Science, Vol. 282, 16 Oct. 1998.

One possible industrial use of ionic ferrofluid slugs isolated by slugs of oil according to the present invention is therefore the external control of liquid slugs within a lab-on-a-chip type microchannel system where a biochemical reaction such as PCR is, for example, carried out in series in each of the aqueous slugs and in parallel over several microchannels.

Other features and advantages of the present invention will become clear on reading the following illustrative but non-limiting examples which make reference to the appended figures.

### BRIEF DESCRIPTION OF THE FIGURES

FIG. 1 is a schematic representation of a microfluidic system according to the present invention comprising a single ferrofluid train;

FIG. 2 is a schematic representation of a microfluidic system according to the present invention in which the magnet system is a permanent magnet;

FIG. 3 is a schematic representation of a microfluidic system according to the present invention in which the magnet system is an electromagnet;

FIGS. 4a to 4c are schematic representations of microfluidic systems according to the present invention in which several applications of the present invention are presented;

FIGS. 5a and 5b are graphical numerical modelling plots showing the flow velocity as a function of time in a 500 μm diameter capillary channel during the passage of a 2 mm long slug of ferrofluid through the magnetic field. The static magnetic field is generated either by two opposing permanent magnets (FIG. 5a) or by a solenoid (FIG. 5b); the origin of the time axis is arbitrary;

FIGS. 6a and 6b are photographs showing an example of the method of the present invention, these photographs being taken against millimeter graph paper in order to demonstrate the scale of the capillary channel and the fluid slugs.

### EXAMPLES

### Example 1

### Ferrofluid Train

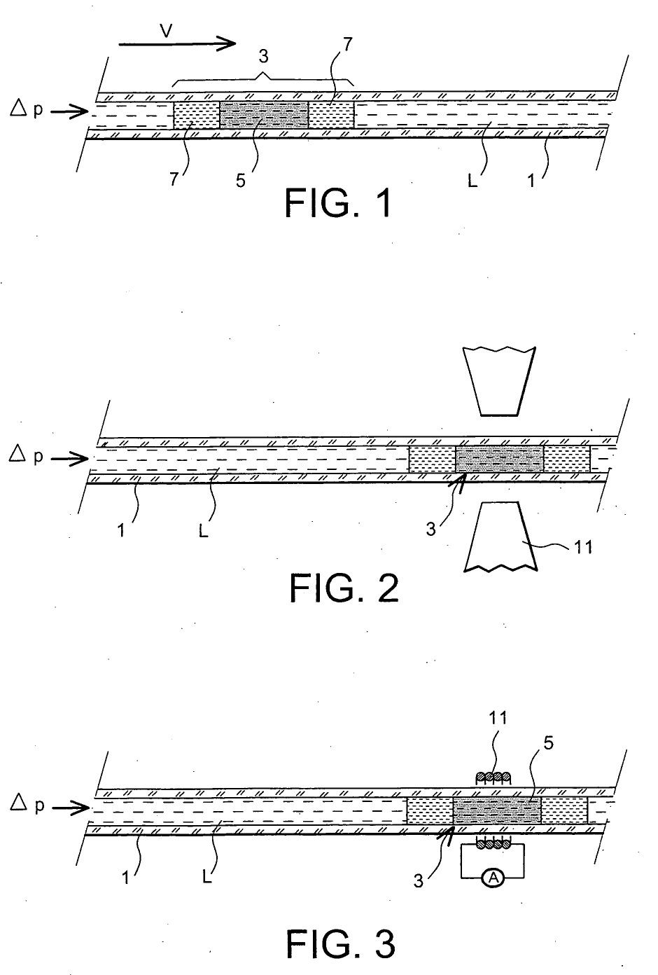

In this example represented in FIG. 1, the ferrofluid train (3) comprises a ferrofluid slug (5) with two slugs (7) of a liquid immiscible with both the ferrofluid and the analyte fluid.

The ferrofluid slug is a slug of ionic ferrofluid containing 20% by weight of magnetic maghemite particles coated with nitrate groups and dispersed in water. The mean particle diameter is equal to 7.5 mm.

The liquid immiscible with both the ferrofluid and the analyte fluid (7) consists of M3516 oil marketed by Sigma-Aldrich.

The capillary channel (1), having a diameter of 500 μm, is made of glass.

In this example, the ferrofluid train is 2 mm in length.

The appended FIG. 2 shows the same capillary channel with a magnet system (11) comprising a permanent magnet in the form of magnetised bars.

FIG. 3 shows the same capillary channel with a magnet system (11) comprising an electromagnet in the form of a solenoid.

This configuration of the microfluidic system of the present invention permits the fluid flow, having a velocity V indicated by the arrow in the capillary channel or microchannel, to be blocked or unblocked. This flow is driven by an externally applied pressure Δp. By retracting the permanent magnets or switching off the electric current, the flow is restored.

### Example 2

### “N Stage” Microfluidic System

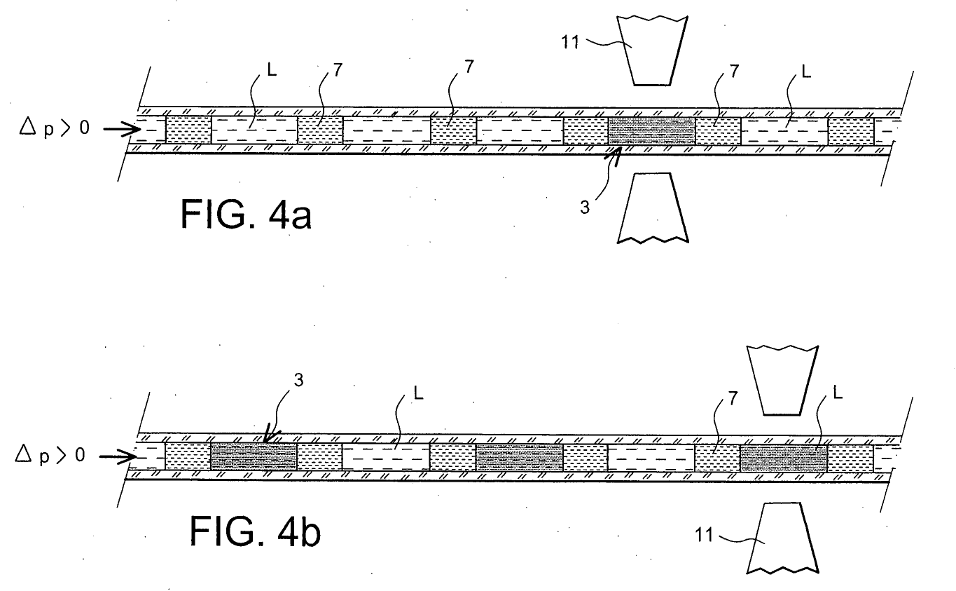

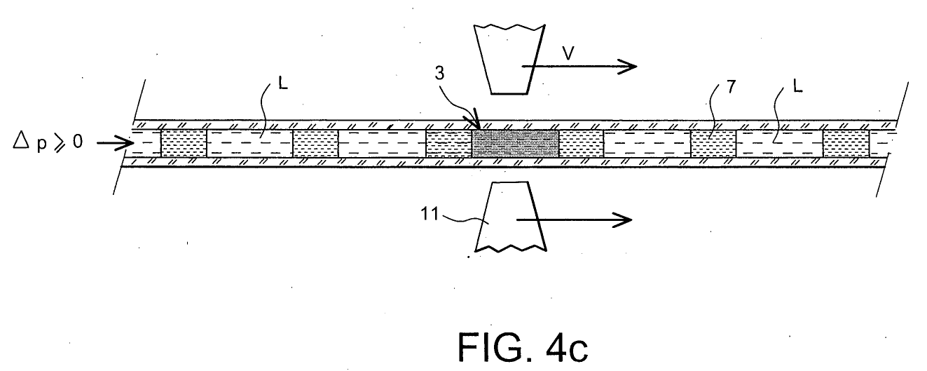

In this example, the same ferrofluid train as used in Example 1 is used in the various applications shown schematically in FIGS. 4a and 4b.

A first application is represented in FIG. 4a. In this application, a single ferrofluid train (3) is used together with several mineral oil slugs (7). In this way, an alternation of analyte fluid (L) and oil slugs (7) precede a ferrofluid train (3).

A second application is represented in FIG. 4b. In this application, several ferrofluid trains (3) are used in alternation with several slugs of analyte fluid (L).

In these two applications, an applied pressure Δp causes the slugs of fluid (L) to flow through the capillary channel. As in Example 1, the magnet system (11) allows this flow to be blocked or unblocked.

This example demonstrates how the extra oil slugs, without slugs of ferrofluid, are used to isolate, for example, biochemical reagents one slug from another.

In these applications, the flow can be sequentially brought to a halt each time a ferrofluid slug passes under a magnet. This configuration allows accurate positioning of the various liquid slugs to be achieved.

### Example 3

### “Continuous Flow” Microfluidic System

In this example, the same ferrofluid train as used in Example 1 is used in the application depicted schematically in FIG. 4c.

This application differs from that shown in FIG. 4a in that the magnet system can move in the direction indicated by the arrows in that figure.

In this application, the displacement of the magnetic field provides the driving force for the displacement of the ferrofluid train within the capillary system, that is to say also the displacement of the analyte fluid (L). The application of a driving pressure is not therefore required here.

### Example 4

### Numerical Modelling

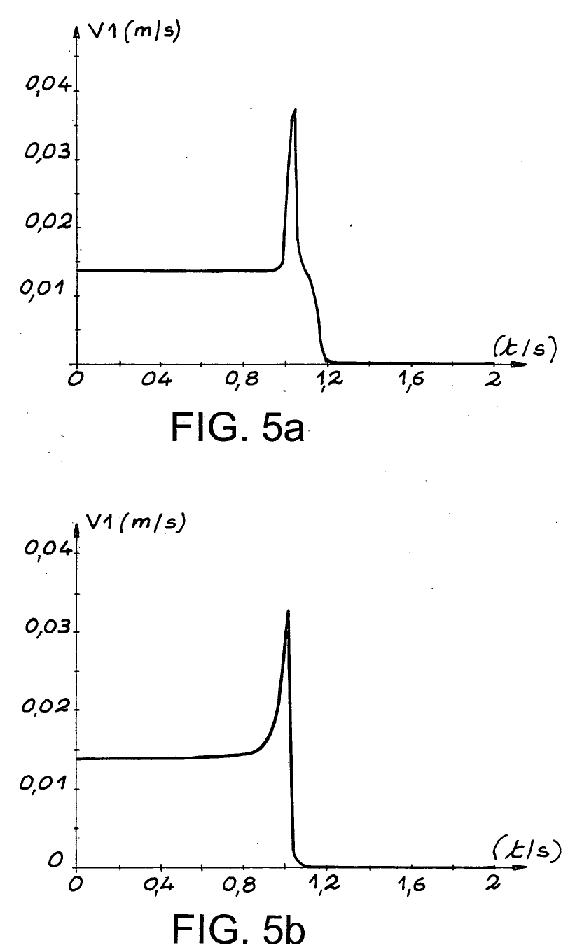

In the appended FIGS. 5a and 5b, numerical simulations using the software package Matlab (registered trademark) show, for example, the arrest of the flow in a capillary channel comprising a succession of ferrofluid trains (as in FIG. 4b) and water.

The magnetic field is created either by two permanent magnets (FIG. 5a) set in opposition or by a solenoid (FIG. 5b). In both cases, magnets or solenoid, the field strength is 350 gauss on the central axis of the capillary channel. The solenoid is 1 mm in diameter and has 10 turns and its length is that of a ferrofluid slug, i.e. 2 mm. The dimensions of the two facing permanent magnets are 3 cm×1 cm×1 mm.

The other parameters used in the numerical simulation are given in the following table:

| | |
|---|---|
| Capillary channel diameter (μm) | 500 |
| Driving pressure (Pa) | 2800 |
| Capillary channel length (m) | 9.6 × 10⁻² |
| Slug lengths (m) | 2 × 10⁻³ |
| Water viscosity (kg/m²s) | 10⁻³ |
| Oil viscosity (kg/m²s) | 3 × 10⁻³ |

### Example 5

### Hydrophobic Capillary Channel

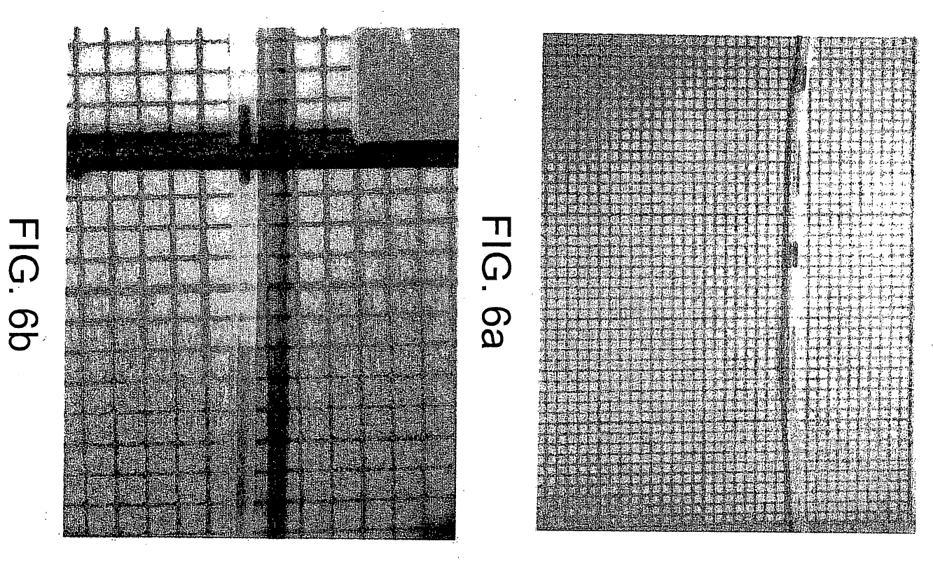

FIGS. 6a and 6b are photographs showing an example of the implementation of the method of the present invention in a capillary microchannel made of Teflon (registered trademark) and with a diameter of 300 μm. Slugs of colourless mineral oil (reference Sigma-Aldrich M3516) are used on either side of a slug of ionic ferrofluid, such as that described in Example 1, to avoid contamination with the slugs of aqueous phase (analyte fluid) that are coloured with methylene blue.

The application of a neodymium-iron-boron bar magnet of dimensions 1×5×36 mm above the microchannel allows the slugs, and hence the flow within the capillary channel, to be controlled externally with a precision of less than 200 μm.

While the same experiment with a glass capillary showed some contaminating ferrofluid deposits on the internal wall of the capillary channel and in the aqueous phase following the passage of the ferrofluid train, no contamination of either the aqueous phase or the internal capillary channel wall with the Teflon coating was observed.

1. Method for the displacement of an analyte fluid within a capillary channel comprising the following steps:
at least one ferrofluid train is introduced into the said microchannel, the said ferrofluid train comprising a slug of ferrofluid and, placed against at least one of the two ends of the slug of ferrofluid and in contact with it, a slug of liquid immiscible with both the ferrofluid and the analyte fluid,
the said analyte fluid is introduced into the said capillary channel in proximity to the ferrofluid train on the side having the slug of liquid immiscible with both the ferrofluid and the analyte fluid, and
displacement of the analyte fluid within the said capillary channel is controlled by the action of a magnetic field on the said slug of ferrofluid, which field is generated by a magnet system placed on the outside of the said capillary channel.

2. Method according to claim 1, in which the ferrofluid is an ionic ferrofluid.

3. Method according to claims 1 or 2, in which the capillary channel is a capillary channel whose internal wall is hydrophobic.

4. Method according to claim 1, in which the capillary channel has a diameter of less than 1 mm.

5. Method according to claim 1, furthermore comprising a step of prewetting the capillary channel internal wall with oil prior to the introduction of the ferrofluid train into the said capillary channel.

6. Method according to claim 1, in which a slug of liquid immiscible with both the ferrofluid and the analyte fluid is placed at each end of the ferrofluid slug.

7. Method according to claim 1, in which a plurality of ferrofluid trains are introduced into the capillary channel.

8. Method according to claim 1, in which at least one slug of liquid immiscible with both the ferrofluid and the analyte fluid is introduced into the capillary channel in between two slugs of analyte fluid.

9. Microfluidic system for the displacement of an analyte fluid comprising, on the one hand, a capillary channel (1) into which at least one ferrofluid train (3) is introduced and, on the other hand, external to the said capillary channel, a magnet system (11) capable of producing a magnetic field for controlling the displacement of the ferrofluid train within the capillary channel, the said ferrofluid train (3) comprising a slug of ferrofluid (5) and, placed against at least one of the two ends of the ferrofluid slug and in contact with it, a slug of liquid (7) immiscible with both the ferrofluid and the analyte fluid.

10. Microfluidic system according to claim 9, in which the ferrofluid is an ionic ferrofluid.

11. Microfluidic system according to claim 9 or 10, in which the capillary channel is a capillary channel whose internal wall is hydrophobic.

12. Microfluidic system according to claim 9, in which the capillary channel has a diameter of less than 1 mm.

13. Microfluidic system according to claim 9, in which a slug of liquid immiscible with both the ferrofluid and the analyte fluid is placed at each end of the ferrofluid slug.

14. Microfluidic system according to claim 9, comprising a plurality of ferrofluid trains.

15. Microfluidic system according to claim 9, in which at least one slug of liquid immiscible with both the ferrofluid and the analyte fluid is introduced into the capillary channel in between two slugs of analyte fluid.

16. Use of a microfluidic system according to claim 9 within an automated in vitro diagnostic system, or within a system for the detection of biological contaminants.
